(19) United States
(12) Patent Application Publication
(10) Pub. No.: US 2010/0280379 A1
(43) Pub. Date: Nov. 4, 2010

(54) ULTRASONIC DIAGNOSTIC APPARATUS

(75) Inventor: Tomoo SATOH, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/552,147

(22) Filed: Sep. 1, 2009

(30) Foreign Application Priority Data
Sep. 3, 2008 (JP) 2008-225415

(51) Int. Cl. A61B 8/14

(52) U.S. Cl. 600/447

(57) ABSTRACT

An ultrasonic diagnostic apparatus in which an image generation time can be reduced and a frame rate can be improved even in a color mode in which a two-dimensional color Doppler image obtained due to Doppler effect is combined with a B-mode image. The ultrasonic diagnostic apparatus includes an ultrasonic probe, a transmission system signal processing unit for supplying drive signals to the ultrasonic probe to transmit pulse trains, each including plural pulses respectively having frequency components of orthogonal frequency division multiplexing waves orthogonal to one another and having different center frequencies from one another, in the same direction, and a reception system signal processing unit for pulse-compressing the plural pulses having different frequency components included in each reception signal outputted from the ultrasonic probe which has received the pulse trains from the same direction, and generating a B-mode image signal based on the compressed pulse.

### CROSS-REFERENCE TO RELATED APPLICATION

The present application claims priority from Japanese Patent Application No. 2008-225415 filed on Sep. 3, 2008, the contents of which are incorporated herein by reference in their entirety.

### BACKGROUND OF THE INVENTION

1. Field of the Invention

The present invention relates to an ultrasonic diagnostic apparatus for imaging organs within a living body by transmitting and receiving ultrasonic waves to generate ultrasonic images to be used for diagnoses.

2. Description of a Related Art

In an ultrasonic diagnostic apparatus for medical use, typically, an ultrasonic probe including plural ultrasonic transducers having functions of transmitting and receiving ultrasonic waves is used. An object to be inspected is scanned with an ultrasonic beam transmitted from the plural ultrasonic transducers, ultrasonic echoes reflected within the object are received by the plural ultrasonic transducers, and thereby, image information (B-mode image) on tissues of the object is obtained based on intensity of the ultrasonic echoes. Further, information (Doppler image) on movement of blood within the object is obtained based on information on frequency shift due to the Doppler effect in the ultrasonic echoes.

In a color mode (color flow mapping mode), a two-dimensional color Doppler image obtained due to the

Doppler effect is combined with a typical B-mode image, and the combined image is displayed. For this purpose, on the way of the transmission for acquiring the B-mode image, transmission for acquiring the Doppler image is performed, and therefore, the number of transmissions per one frame increases and the frame rate decreases. Further, in order to secure the sensitivity in detection of the Doppler effect, plural times (four to ten times) of transmissions are performed in the same direction, and the spatial resolving power becomes poor. Accordingly, reduction in image generation time is desired in the color mode.

As a related technology, Japanese Patent Application Publication JP-P2004-321647A discloses an ultrasonic diagnostic apparatus intended to reduce the image generation time, further improve an S/N ratio of the image signal, and thereby, clearly display a minute object or moving object for accurate observation of a minute part. The ultrasonic diagnostic apparatus for generating transmission ultrasonic beams and reception ultrasonic beams by dynamic focusing by using plural transmission signals at different frequencies and generating an image by electrically scanning with these ultrasonic beams, includes (a) transmission signal generating means for setting frequencies, amplitude and forms such that, at center frequencies of frequency spectra of the respective transmission signals, levels of the frequency spectra of the other transmission signals becomes at equal to or less than a predetermined level, and generating these plural transmission signals at predetermined intervals in descending order of frequency within a transmission permissible time at every transmission and reception repetition period, (b) transmission beam forming means for forming the transmission ultrasonic beams by dynamic focusing by transmitting the respective transmission signals generated by the transmission signal generating means, (c) frequency analyzing means for generating the reception ultrasonic beams by dynamic focusing with respect to reception signals received when the respective transmission signals generated by the transmission signal generating means are transmitted, and frequency-analyzing output of the reception ultrasonic beams to extract center frequency components of the respective transmission signals and arrange them in chronological order, and (d) a correlation processing unit for performing cross-correlation processing on the signals in the respective time series extracted by the frequency analyzing means to generate an image signal on one ultrasonic line.

However, JP-P2004-321647A does not especially disclose the color mode. Further, Edition of Electronic Industries Association of Japan, “Handbook of Ultrasonic Diagnostic Equipments”, Revised Version, CORONA Publishing, Jan. 20, 1997, pp. 118-131 discloses a general pulse Doppler apparatus.

### SUMMARY OF THE INVENTION

The present invention has been achieved in view of the above-mentioned points. A purpose of the present invention is to provide an ultrasonic diagnostic apparatus in which an image generation time can be reduced and a frame rate can be improved even in a color mode in which a two-dimensional color Doppler image obtained due to Doppler effect is combined with a B-mode image.

In order to accomplish the above-mentioned purpose, an ultrasonic diagnostic apparatus according to one aspect of the present invention includes: an ultrasonic probe including plural ultrasonic transducers for transmitting ultrasonic waves according to drive signals and receiving ultrasonic echoes to output reception signals; transmission system signal processing means for supplying drive signals to the ultrasonic probe to transmit pulse trains, each including plural pulses respectively having frequency components of orthogonal frequency division multiplexing waves orthogonal to one another and having different center frequencies from one another, in the same direction ; and reception system signal processing means for pulse-compressing the plural pulses having different frequency components included in each reception signal outputted from the ultrasonic probe which has received the pulse trains from the same direction, and generating a B-mode image signal based on a compressed pulse.

According to the one aspect of the present invention, by transmitting and receiving the pulse trains, each including plural pulses respectively having frequency components of orthogonal frequency division multiplexing waves, in the same direction, plural pulses are pulse-compressed to generate a B-mode image signal and the plural pulses can also be used for generation of a Doppler image signal. Therefore, even in the color mode, the image generation time can be reduced and the frame rate can be improved.

### BRIEF DESCRIPTION OF THE DRAWINGS

FIG. 1 is a block diagram showing a configuration of an ultrasonic diagnostic apparatus according to the first embodiment of the present invention;

FIG. 2 shows a configuration example of a pulse compressing unit shown in FIG. 1;

FIG. 3 shows waveforms of a pulse train in position A shown in FIG. 2;

FIG. 4 shows waveforms of plural pulses in position B shown in FIG. 2;

FIG. 5 is a diagram for explanation of pulse compression on a frequency axis;

FIG. 6 specifically shows a spectrum of a compressed pulse;

FIG. 7 is a diagram for explanation of interpolation of frequency components;

FIG. 8 shows a configuration example of a Doppler detecting unit shown in FIG. 1;

FIG. 9 is a diagram for detailed explanation of an operation of the Doppler detecting unit shown in FIG. 8;

FIG. 10 shows Q signals and I signals obtained by orthogonal detection of the pulse trains shown in FIG. 9;

FIGS. 11A-11C show phase shifts obtained based on the Q signals and I signals shown in FIG. 10; and

FIG. 12 is a block diagram showing a configuration of an ultrasonic diagnostic apparatus according to the second embodiment of the present invention.

### DESCRIPTION OF THE PREFERRED EMBODIMENTS

Hereinafter, embodiments of the present invention will be explained in detail with reference to the drawings. The same reference numerals are assigned to the same component elements and the explanation thereof will be omitted.

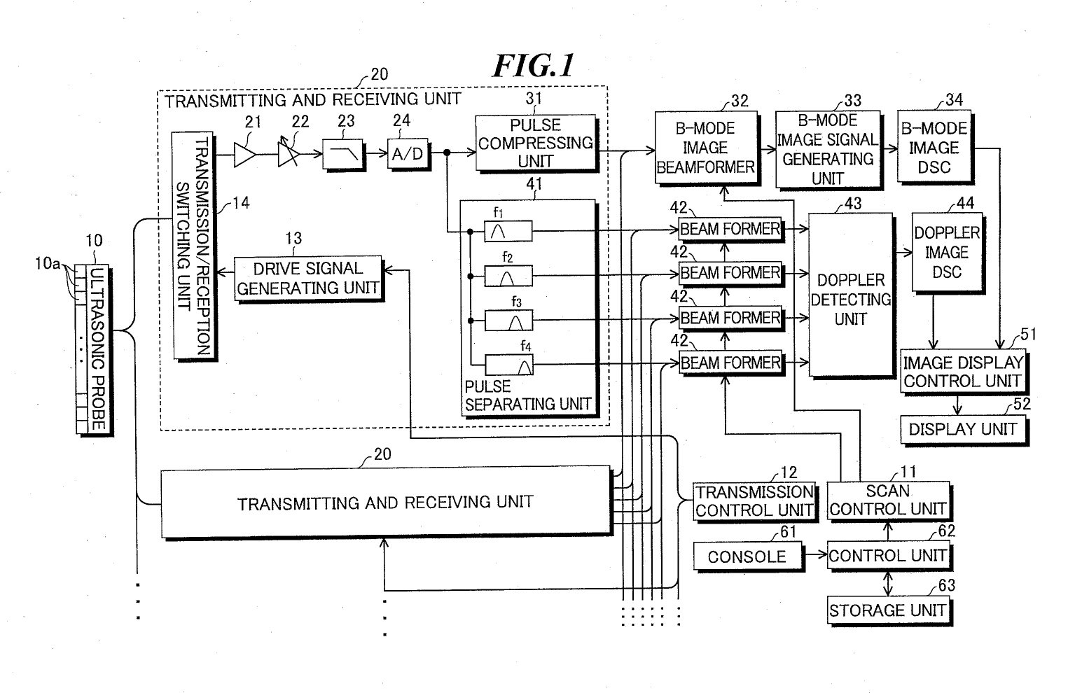

FIG. 1 is a block diagram showing a configuration of an ultrasonic diagnostic apparatus according to the first embodiment of the present invention. The ultrasonic diagnostic apparatus has an ultrasonic probe 10 including plural ultrasonic transducers 10a, a scan control unit 11, a transmission control unit 12, transmitting and receiving units 20 having plural channels corresponding to the plural ultrasonic transducers 10a, a B-mode image beamformer 32, a B-mode image signal generating unit 33, a B-mode image DSC 34, Doppler image beam formers 42, a Doppler detecting unit 43, a Doppler image DSC 44, an image display control unit 51, a display unit 52, a console 61, a control unit 62, and a storage unit 63.

The plural ultrasonic transducers 10a of the ultrasonic probe 10 transmit ultrasonic waves to an object to be inspected according to applied drive signals, and receive ultrasonic echoes propagating from the object to output reception signals. These ultrasonic transducers 10a are one-dimensionally or two-dimensionally arranged to form a transducer array.

Each ultrasonic transducer includes a vibrator having electrodes formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride),or the like. When a pulsed or continuous wave voltage is applied to the electrodes of the vibrator, the piezoelectric material expands and contracts. By the expansion and contraction, pulse or continuous wave ultrasonic waves are generated from the respective vibrators, and an ultrasonic beam is formed by synthesizing these ultrasonic waves. Further, the respective vibrators expand and contract by receiving the propagating ultrasonic waves to generate electric signals. These electric signals are outputted as reception signals of ultrasonic waves.

The scan control unit 11 can set the transmission direction of the ultrasonic beam transmitted from the ultrasonic probe 10, reception direction, focal depth, and an aperture diameter of the ultrasonic transducer array when an imaging region within the object is scanned with the ultrasonic beam. The scan control unit 11 controls the transmission control unit 12, the B-mode image beamformer 32, and the Doppler image beam formers 42 according to the settings.

The transmission control unit 12 sets delay times to be respectively provided to the drive signals for the plural ultrasonic transducers 10a based on a transmission delay pattern according to the transmission direction set by the scan control unit 11. Alternatively, the transmission control unit 12 may set delay times such that ultrasonic waves transmitted from the plural ultrasonic transducers 10a at the same time reach the imaging region of the object.

The transmitting and receiving unit 20 has plural channels corresponding to the plural ultrasonic transducers 10a. Each channel of the transmitting and receiving unit 20 includes a drive signal generating unit 13, a transmission/reception switching unit 14, a preamplifier 21, a variable gain amplifier 22, a low-pass filter 23, an A/D converter 24, a pulse compressing unit 31, and a pulse separating unit 41.

The drive signal generating unit 13 includes a pulser or the like for generating a drive signal to be supplied to the corresponding ultrasonic transducer 10a according to the delay time set by the transmission control unit 12. The plural drive signal generating units 13 may adjust the amounts of delay of the drive signals and supply the drive signals to the ultrasonic probe 10 such that the ultrasonic waves transmitted from the plural ultrasonic transducers 10a form an ultrasonic beam, or may supply the drive signals to the ultrasonic probe 10 such that the ultrasonic waves transmitted from the plural ultrasonic transducers 10a at the same time reach the entire imaging region of the object.

Here, the transmission control unit 12 and the plural drive signal generating units 13 form transmission system signal processing means, and supply drive signals to the ultrasonic probe 10 to transmit pulse trains in the same direction. Each pulse train includes plural pulses respectively having frequency components of orthogonal frequency division multiplexing (OFDM) waves orthogonal to one another and having different center frequencies from one another. One pulse train includes N pulses generated at predetermined pulse intervals (N is an integer number equal to or more than “2”).

According to the embodiment, plural pulses respectively having orthogonal frequency components having different center frequencies are compressed, and thereby, a B-mode image signal is obtained. Further, a Doppler image signal is obtained by orthogonal detection of the plural pulses. Therefore, the image generation time can be reduced and the frame rate can be improved.

The transmission/reception switching unit 14 switches between output of the drive signal to the ultrasonic probe 10 and input of the reception signal from the ultrasonic probe 10. The reception signal outputted from the ultrasonic transducer 10a receiving the ultrasonic echoes reflected within the object is amplified by the preamplifier 21 and the variable gain amplifier 22, band-limited by the low-pass filter 23, and converted into a digital reception signal by the A/D converter 24. The A/D converter 24 supplies the digital reception signal to the pulse compressing unit 31 and the pulse separating unit 41.

The pulse compressing unit 31 pulse-compresses the plural pulses having different frequency components included in the corresponding reception signal outputted from the ultrasonic probe 10 that has received the pulse trains from the same direction. Each pulse train includes plural pulses respectively having frequency components of orthogonal frequency division multiplexing waves orthogonal to one another and having different center frequencies from one another.

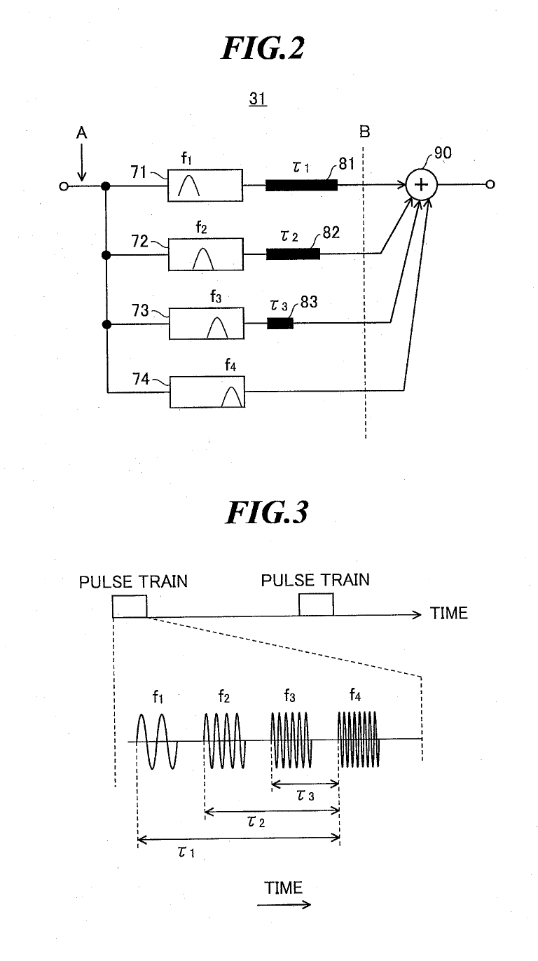

FIG. 2 shows a configuration example of the pulse compressing unit in FIG. 1. As below, the case where one pulse train includes four pulses will be explained (N=4). The i-th pulse includes a pulse-modulated sine wave having a frequency of fᵢ (i=1, 2, 3, 4). The pulse compressing unit 31 is equivalently expressed by four bandpass filters 71-74 having center pass frequencies of f₁-f₄, respectively, three delay elements 81-83 providing delay times of τ₁-τ₃, respectively, and an adder 90 for adding the four frequency components to one another. Here, the delay times of τ₁-τ₃ correspond to the pulse intervals in the pulse train.

FIG. 3 shows waveforms of the pulse train in position A in FIG. 2. As shown in FIG. 3, one pulse train includes the first to fourth pulses having center frequencies of f₁-f₄, respectively, arranged in chronological order at the predetermined pulse intervals. Here, the pulse interval between the first pulse and the fourth pulse is τ₁, the pulse interval between the second pulse and the fourth pulse is τ₂, and the pulse interval between the third pulse and the fourth pulse is τ₃.

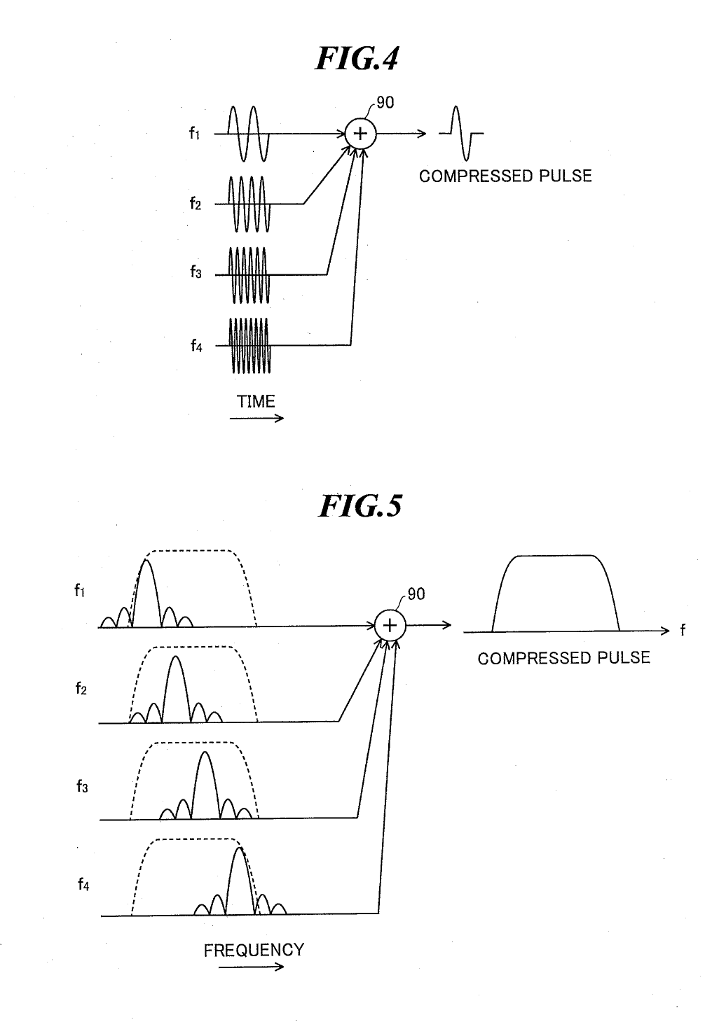

FIG. 4 shows waveforms of the plural pulses in position B in FIG. 2. By using the bandpass filters 71-74 as shown in FIG. 2, the first to fourth pulses including frequency components having center frequencies of f₁-f₄, respectively, are extracted. By using the delay elements 81-83 as shown in FIG. 2, the first to fourth pulses are time-aligned. By using the adder 90, the first to fourth pulses are added to one another. In this way, pulse compression is performed, and a compressed single pulse is generated.

FIG. 5 is a diagram for explanation of pulse compression on a frequency axis. As shown in FIG. 5, by using the adder 90, the narrowband frequency components having the center frequencies of f₁-f₄ are added to one another, and a compressed pulse having wideband frequency components is generated.

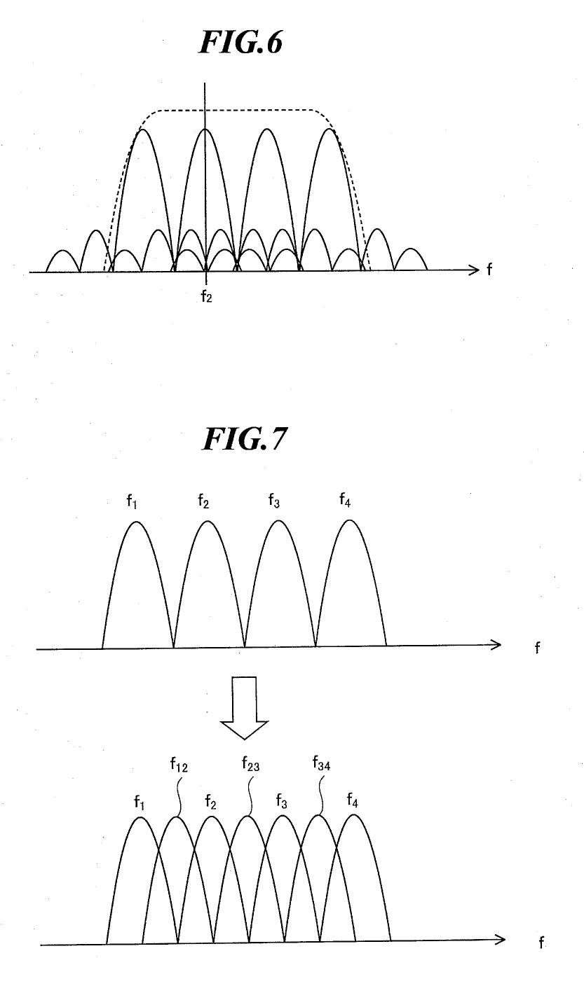

FIG. 6 specifically shows a spectrum of the compressed pulse. The first to fourth frequency components having center frequencies of f₁-f₄, respectively, are orthogonal to one another, and therefore, at the center frequency (peak frequency) f₂ of the second frequency component, for example, amplitude of the other frequency components is zero.

However, in the case where the number of frequency components to be combined is smaller, information for forming a single pulse having wideband frequency components is insufficient. In such a case, when plural pulses having different frequency components included in the reception signal are pulse-compressed, the pulse compressing unit 31 shown in FIG. 1 may interpolate the frequency components not included in the reception signal.

FIG. 7 is a diagram for explanation of interpolation of frequency components. As shown in FIG. 7, three frequency components having center frequencies of f₁₂, f₂₃, f₃₄ are interpolated between the four frequency components having center frequencies of f₁-f₄, and thereby, missing information is supplemented and the spectrum continues smoothly. The interpolation can be realized, for example, by performing Fourier transform on the reception signal, adding a desired frequency component on the frequency axis based on the four frequency components having center frequencies of f₁-f₄, and then, performing inverse Fourier transform thereon.

Referring to FIG. 1 again, the B-mode image beamformer 32 has plural delay patterns (phase matching patterns) according to reception directions and focal depths of ultrasonic echoes, andperforms reception focusing processing by providing the respective delays to the plural pulses respectively compressed by the pulse compressing units 31 of the plural channels according to the reception direction and focal depth set by the scan control unit 11 and adding the plural pulses to one another. By the reception focusing processing, a compressed pulse (B-mode sound ray signal), in which the focus of the ultrasonic echoes is narrowed, is formed.

The B-mode image signal generating unit 33 performs envelope detection processing on the B-mode sound ray signal, and further performs preprocess processing such as Log (logarithm) compression and gain adjustment to generate a B-mode image signal. The B-mode image DSC 34 converts (raster-converts) the generated B-mode image signal into an image signal that follows the normal scan system of television signals. In the above description, the pulse compressing unit 31 to B-mode image DSC 34 form first reception system signal processing means.

The pulse separating unit 41 includes four bandpass filters having center pass frequencies of f₁-fₒᵣ respectively, and separates four kinds of pulses having the center frequencies of f₁-f₄, respectively. The four kinds of pulses separated by the pulse separating unit 41 of each channel are supplied to the four Doppler image beam formers 42, respectively.

Each Doppler image beam former 42 has plural delay patterns (phase matching patterns) according to reception directions and focal depths of ultrasonic echoes, and performs reception focusing processing by providing the respective delays to the plural pulses respectively outputted from the pulse separating units 41 of the plural channels according to the reception direction and focal depth set by the scan control unit 11 and adding the plural pulses to one another. By the reception focusing processing, pulses (Doppler sound ray signals), in which the focus of the ultrasonic echoes is narrowed, are formed.

The Doppler detecting unit 43 performs orthogonal detection by multiplying frequency components of the respective pulses supplied from the four Doppler image beam formers 42 by local oscillation signals having corresponding center frequencies to generate baseband signals. Further, the Doppler detecting unit 43 detects frequency shifts in the respective pulses based on phase information of the base band signals to generate a Doppler image signal.

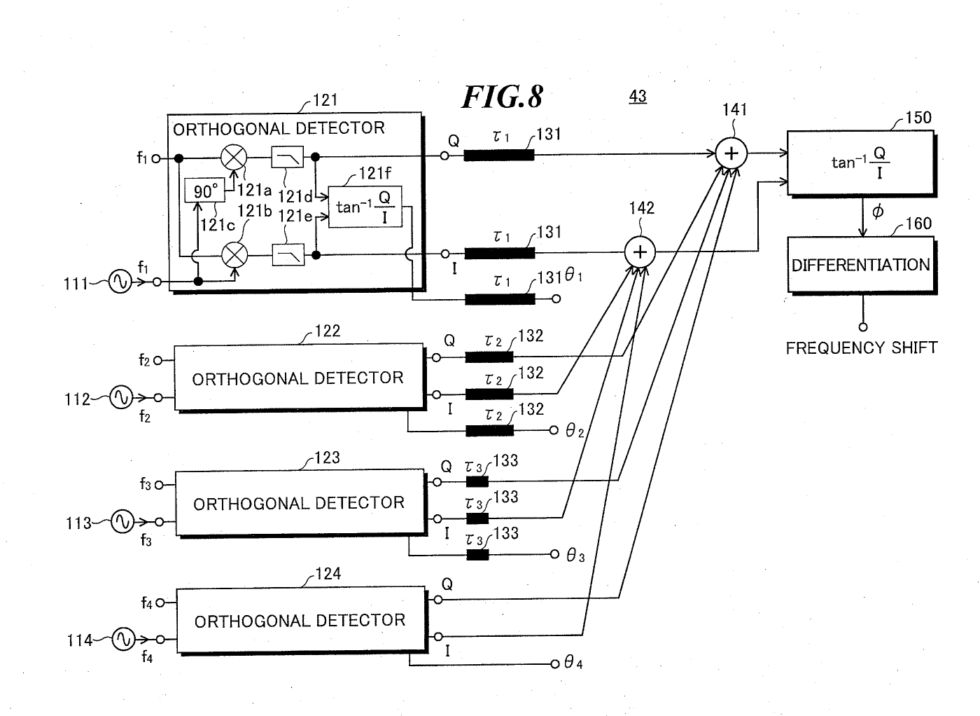

FIG. 8 shows a configuration example of the Doppler detecting unit in FIG. 1. The Doppler detecting unit 43 is equivalently expressed by four local oscillators 111-114 having oscillation frequencies of f₁-f₄, respectively, four orthogonal detectors 121-124, three kinds of delay elements 131-133 providing delay times of τ₁-τ₃, respectively, an adder 141 for adding four Q signals to one another, an adder 142 for adding four I signals to one another, a phase computing part 150 for computing a phase of the baseband signal based on the Q signals and the I signals after addition, and a differential computing part 160 for computing a frequency shift by differentiating the phase of the baseband signal.

The orthogonal detector 121 is equivalently expressed by two mixers (multipliers) 121a and 121b, a 90° phase shifter 121c, two low-pass filters 121d and 121e, and a phase computing part 121f.

The frequency component having a center frequency of f₁ and supplied from the first Doppler image beam former 42 is multiplied by an oscillation signal of the local oscillator 111 in the mixer 121a, and supplied to the low-pass filter 121d. Further, the phase of the oscillation signal of the local oscillator 111 is rotated by 90° in the 90° phase shifter 121c. The frequency component having the center frequency of f₁ and supplied from the first Doppler image beam former 42 is multiplied by the oscillation signal having a phase rotated by 90° in the mixer 121b, and supplied to the low-pass filter 121e.

Thereby, orthogonal detection is performed, and a Q signal (imaginary component) and an I signal (real component) forming a complex baseband signal are outputted from the low-pass filters 121d and 121e, respectively. The phase computing part 121f performs computation of tan ⁻¹ (Q/I) based on the Q signal and the I signal to obtain a phase shift signal representing a phase shift of the complex baseband signal.

The configurations and operations of the orthogonal detectors 122-124 are the same as those of the orthogonal detector 121. The phase shift signals outputted from the orthogonal detectors 121-123 are inputted to the delay elements 131-133, respectively, and four kinds of phase shift signals θ₁-θ₄ are time-aligned. Further, the phase shift signals are differentiated and frequency shift signals representing the frequency shifts in the plural pulses are obtained. Then, a Doppler image signal representing a velocity of a mobile unit within the object is obtained based on the frequency shift signals.

On the other hand, the Q signals and I signals outputted from the orthogonal detectors 121-123 are inputted to the delay elements 131-133, respectively, and four kinds of Q signals and I signals are time-aligned. The adder 141 adds the four kinds of Q signals to one another, and the adder 142 adds the four kinds of I signals to one another, and thereby, the detection accuracy when the frequency shift is computed can be improved. The phase computing part 150 performs computation of tan ⁻¹ (Q/I) based on the Q signals and the I signals after addition, and obtains an average phase shift signal φ averagely representing the phase shifts in the four kinds of pulses. Further, the differential computing part 160 differentiates the average phase shift signal φ to obtain a frequency shift signal representing the frequency shift in the plural pulses, and furthermore, obtains a Doppler image signal representing a velocity of a mobile unit within the object based on the frequency shift signal.

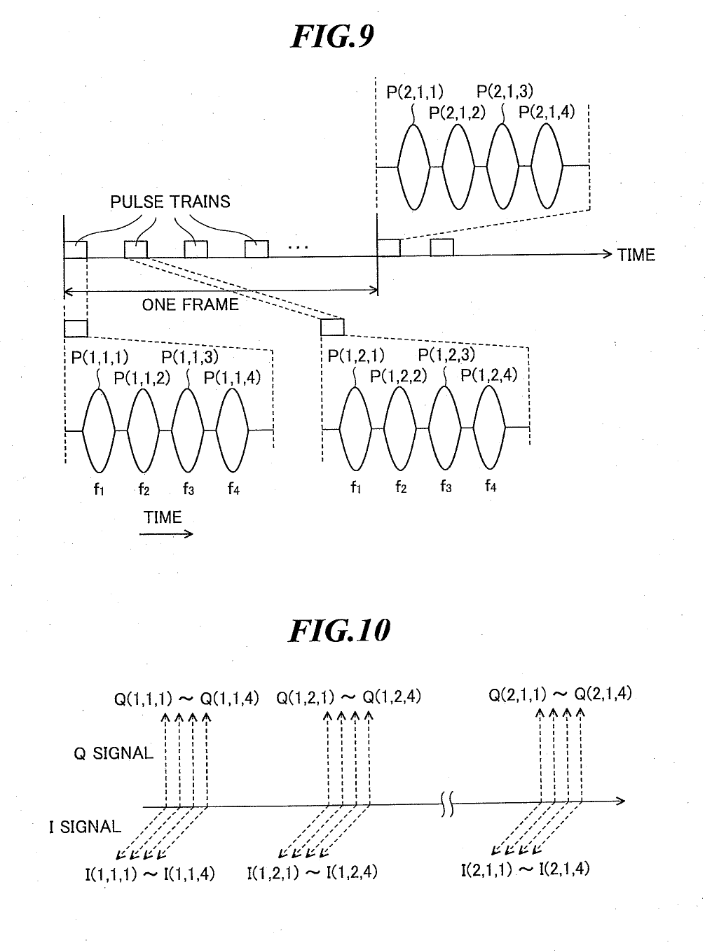

FIG. 9 is a diagram for detailed explanation of an operation of the Doppler detecting unit as shown in FIG. 8. As shown in FIG. 9, plural pulse trains are received in each frame. The pulses included in those pulse trains are expressed by P(i,j,k). Here, “i” represents a frame number, “j” represents a pulse train number in each frame, and “k” represents a pulse number in each pulse train. Further, in each pulse train, the first pulse P(i,j,1) to fourth pulse P(i,j,4) include frequency components having center frequencies of f₁-f₄, respectively.

FIG. 10 shows Q signals and I signals obtained by orthogonal detection of the pulse trains as shown in FIG. 9. By orthogonal detection of the pulse P(i,j,k), Q signal Q(i,j,k) and I signal I(i,j,k) are obtained.

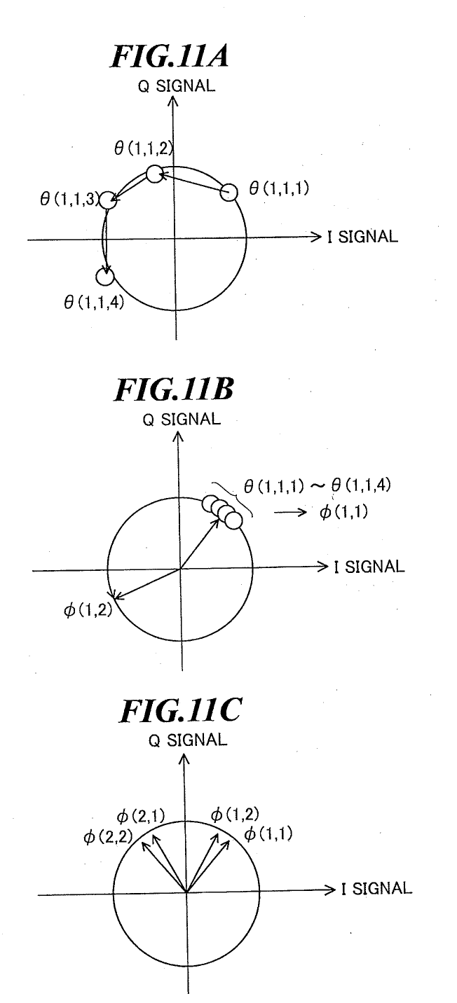

FIGS. 11A-11C show phase shifts obtained based on the Q signals and I signals as shown in FIG. 10. By performing the computation of tan ⁻¹ (Q(i,j,k)/I(i,j,k)) based on the Q signal Q(i,j,k) and the I signal I(i,j,k) as shown in FIG. 10, phase shift signal θ(i,j,k) representing the phase shift in each pulse is obtained.

FIG. 11A shows the case where a high moving velocity (flow velocity) is detected, and the velocity of a mobile unit within the object is detected based on the phase shift signals θ(i,j,1), θ(i,j,2), θ(i,j,3), θ(i,j,4), . . . FIG. 11B shows the case where a general moving velocity (flow velocity) is detected, and the velocity of a mobile unit within the object is detected based on the average phase shift signals φ(i,j) representing an average of the phase shift signals θ(i, j,k) in each pulse train. FIG. 11C shows the case where a low moving velocity (flow velocity) is detected, and the velocity of a mobile unit within the object is detected based on the average phase shift signals φ(1, j), φ(2, j), . . . between plural frames.

Referring to FIG. 1 again, the Doppler image DSC 44 converts (raster-converts) the Doppler image signal generated by the Doppler detecting unit 43 into an image signal that follow the normal scan system of television signals. In the above description, the pulse separating unit 41 to Doppler image DSC 44 form second reception system signal processing means.

The image display control unit 51 combines the image signal supplied from the B-mode image DSC 34 and the image signal supplied from the Doppler image DSC 44, and generates an image signal for display in the color mode.

The console 61 includes a keyboard, an adjustment knob, a mouse, and so on, and is used by an operator for inputting commands and information to the ultrasonic diagnostic apparatus. The control unit 62 controls the respective units of the ultrasonic diagnostic apparatus according to the commands and information inputted by using the console 61. In the embodiment, the scan control unit 11, the transmission control unit 12, the pulse compressing unit 31 to image display control unit 51, and the control unit 62 are formed of a central processing unit (CPU) and software for allowing the CPU to execute various kinds of processing. Alternatively, they may be formed of digital circuits or analog circuits. The software is stored in the storage unit 63. As a recording medium in the storage unit 63, not only a built-in hard disk but also a flexible disk, MO, MT, RAM, CD-ROM, DVD-ROM, or the like may be used.

Next, the second embodiment of the present invention will be explained.

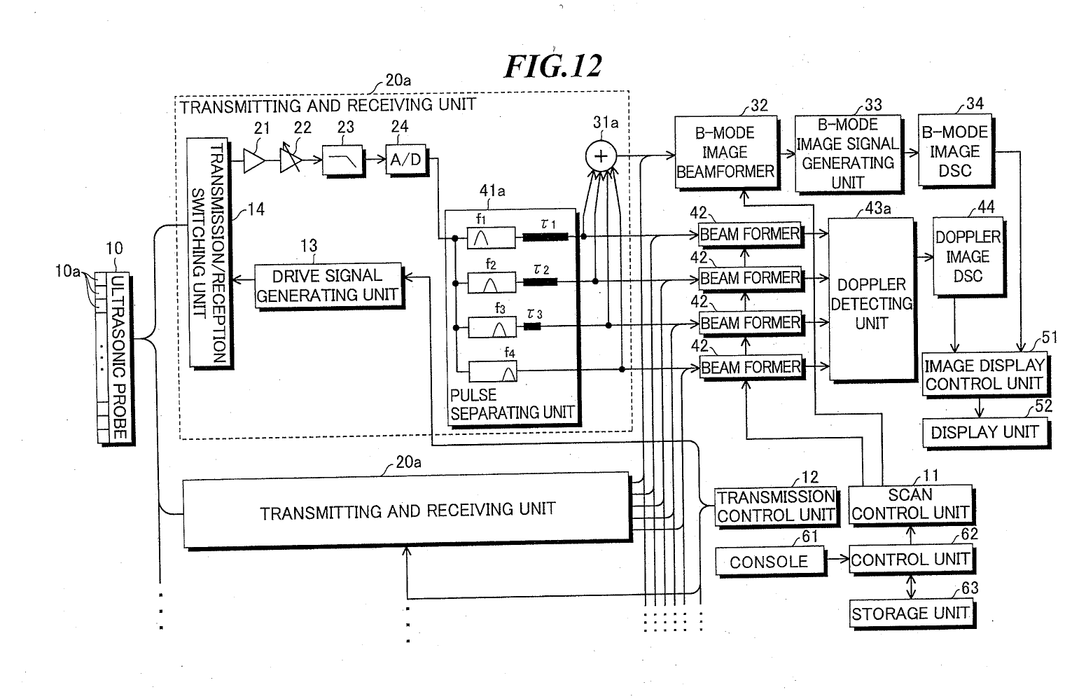

FIG. 12 is a block diagram showing a configuration of an ultrasonic diagnostic apparatus according to the second embodiment of the present invention. In the second embodiment, in place of the transmitting and receiving units 20 and the Doppler detecting unit 43, transmitting and receiving units 20a and a Doppler detecting unit 43a are used. The rest of the configuration is the same as that in the first embodiment.

In the transmitting and receiving unit 20a, a pulse separating unit 41a includes four bandpass filters having center pass frequencies of f₁-f₄, respectively, and additionally includes three delay elements respectively providing delay times of τ₁-τ₃. These may also serve as the bandpass filters 71-74 and the delay elements 81-83 of the pulse compressing unit as shown in FIG. 2. Accordingly, in the second embodiment, a pulse compressing unit 31a includes only an adder. Further, in the Doppler detecting unit 43a, the delay elements 131-133 as shown in FIG. 9 are not necessary.

In the second embodiment, the pulse separating unit 41a, the pulse compressing unit 31a, B-mode image beamformer 32, B-mode image signal generating unit 33, and B-mode image DSC 34 form first reception system signal processing means. Further, the pulse separating unit 41a, the Doppler image beamformers 42, the Doppler detecting unit 43a, and Doppler image DSC 44 form second reception system signal processing means.

1. An ultrasonic diagnostic apparatus comprising:
an ultrasonic probe including plural ultrasonic transducers for transmitting ultrasonic waves according to drive signals and receiving ultrasonic echoes to output reception signals;
transmission system signal processing means for supplying drive signals to the ultrasonic probe to transmit pulse trains, each including plural pulses respectively having frequency components of orthogonal frequency division multiplexing waves orthogonal to one another and having different center frequencies from one another, in a same direction; and
first reception system signal processing means for pulse-compressing the plural pulses having different frequency components included in each reception signal outputted from the ultrasonic probe which has received the pulse trains from the same direction, and generating a B-mode image signal based on a compressed pulse.

2. The ultrasonic diagnostic apparatus according to claim 1, wherein said first reception system signal processing means interpolates, when pulse-compressing the plural pulses having different frequency components included in the reception signal, frequency components not included in the reception signal.

3. The ultrasonic diagnostic apparatus according to claim 1, further comprising:
second reception system signal processing means for separating the plural pulses having different frequency components included in the reception signal by bandpass filter processing, performing reception focusing processing on the respective pulses, and then, performing orthogonal detection by multiplying the frequency components of the plural pulses by local oscillation signals having corresponding center frequencies to generate baseband signals, and detecting frequency shifts in the plural pulses based on phase information of the baseband signals to generate a Doppler image signal.

4. The ultrasonic diagnostic apparatus according to claim 2, further comprising :
second reception system signal processing means for separating the plural pulses having different frequency components included in the reception signal by bandpass filter processing, performing reception focusing processing on the respective pulses, and then, performing orthogonal detection by multiplying the frequency components of the plural pulses by local oscillation signals having corresponding center frequencies to generate baseband signals, and detecting frequency shifts in the plural pulses based on phase information of the baseband signals to generate a Doppler image signal.

5. The ultrasonic diagnostic apparatus according to claim 3, said second reception system signal processing means delays the baseband signals generated by orthogonal detection of the plural pulses, on which reception focusing processing have been performed, according to respective pulse intervals, and adding the baseband signals to one another.

6. The ultrasonic diagnostic apparatus according to claim 4, said second reception system signal processing means delays the baseband signals generated by orthogonal detection of the plural pulses, on which reception focusing processing have been performed, according to respective pulse intervals, and adding the baseband signals to one another.

7. The ultrasonic diagnostic apparatus according to claim 3, said second reception system signal processing means detects the frequency shifts between the plural pulses belonging to different frames based on phase information of the baseband signals generated by orthogonal detection of the plural pulses, on which reception focusing processing have been performed, in plural frames.

8. The ultrasonic diagnostic apparatus according to claim 4, said second reception system signal processing means detects the frequency shifts between the plural pulses belonging to different frames based on phase information of the baseband signals generated by orthogonal detection of the plural pulses, on which reception focusing processing have been performed, in plural frames.
